(19) United States
(12) Patent Application Publication
(10) Pub. No.: US 2004/0018155 A1
(43) Pub. Date: Jan. 29, 2004

(54) Toothpaste formulation

(76) Inventor: Ronald Hoagland, Flanders, NJ (US)

Correspondence Address: BENJAMIN APPELBAUM, Ph.D., Attorney-At-Law, 27 Bennington Drive, Flanders, NJ, 07836-9756, US

(21) Appl. No.: 10/627,887

(22) Filed: Jul. 25, 2003

Related U.S. Application Data
(60) Provisional application No. 60/398,383, filed on Jul. 25, 2002.

(51) Int. Cl. A61K 7/16

(52) U.S. Cl. 424/49

(57) ABSTRACT

A method of preparing for hunting comprises applying a quantity of a toothpaste to the hunter's teeth, the toothpaste formulated to contain no added scents or odors. The formulation provides hunters with the ability to practice their normal tooth brushing routine, but without adding a scent, which if detected by the prey animal, could compromise the hunter's objective of being scent free to avoid detection by the prey while hunting. An embodiment comprises a mixture of calcium carbonate, a polyol; water; sodium bicarbonate; sodium lauryl sulfate; titanium dioxide; guar gum, and sodium saccharin.

### CROSS-REFERENCE TO RELATED APPLICATION

This application claims the benefit of U.S. Provisional patent application Serial No. 60/398,383, filed Jul. 25, 2002, the contents of which are hereby incorporated by reference herein.

### FIELD OF THE INVENTION

The present invention is a toothpaste formulation. In particular, the present invention is a toothpaste formulation intended for use primarily by hunters, the formulation containing ingredients to which no added scent has been added, to avoid the emission of odors from the hunter which may indicate to the prey the presence of something unusual in its environment.

### BACKGROUND OF THE INVENTION

The present invention is intended for the hunting market, consisting of approximately 15,000,000 licensed hunters in the United States. The present invention is intended for hunters that need to exercise human, or foreign, scent control for the best potential of success. This population, among others, can easily be identified as deer hunters. Deer hunters represent 92% of the total US hunting license holders. An embodiment of the present invention can be more narrowly targeted towards the serious deer hunter, which for purposes of the present specification, is considered to be an individual characterized as one who may spend more than 10 days a year afield and who falls within one of the following groups: bow hunters; muzzleloader hunters; shotgun hunters; and those who hunt using special and extended season permits. These individuals regularly purchase scent control, cover, or attractatproducts.

### Hunting Market Products

The national deer hunting market is very large and clearly identifiable by the above criteria. Hunting periodicals have published data indicating that approximately 58% of their readers purchase more than $1,000 per year of the following deer hunting related products.

Major Categories of Products Purchased Annually ($1,000)

Firearms (all types, including bow hunting products)

Clothing

Camouflage

Tree stands

Deer attractant scents

Cover scents (scents to cover-up the scent of the hunter)

Scent control products

Published scientific studies clearly indicate that a deer's sense of smell is the number one means of defense, survival, and procreation.

Primary Senses for Defense, Survival, and Procreation

1. Smell

2. Hearing

3. Sight

The sense of smell is followed closely by hearing as the second most relied upon sense, with sight a distant third. Once something is detected as being “not normal” by one sense, a deer will often try to confirm the source by using a second sense. However, smell does not usually require confirmation unless curiosity becomes a factor. The intensity of the scent detected by the animal can also be an indication of a potential threat; a certain concentration detected by the animal may signal the presence of something within the area, but not at a level to raise alarm. If the scent being detected increases, it could trigger either a perceived threat, and cause the animal to flee, or if the scent is perceived as something innocuous or acceptable to the animal, allow it to go about its routine.

Serious hunters regularly purchase and use many, if not all, of the scent control products listed below but will generally use a “normal consumer” scented/flavored toothpaste to brush their teeth. With the use of the scent control products, literally, a hunter's whole body and equipment will be scent free but their mouth and breath will be “minty fresh”. This minty, or other flavor, breath although not offensive, is not part a deer's natural environment. Data indicate that wary deer avoid any indication of something that is “not normal”. The resulting theory is that anything “foreign” represents a potential threat to deer, and may cause the deer to flee.

The Major Scent Control Product Categories Include Products Such as:

Scent elimination spray

Clothes wash

Hair and body soap

Underarm deodorant

Scent control suits/clothing

This inventor is an avid hunter, and has searched for several years to find an oral care product to use for brushing teeth without scent, or a product to eliminate or cover-up the scent of toothpaste. The only option found for scent free brushing was plain baking soda, which wasn't very pleasant. Although a mouthwash and chewing gum have been reported to have been marketed, these products focused on the elimination of bacteria that cause odor and did not cover-up the scents that are included in conventional toothpaste; this research during recent hunting seasons has indicated that these products are no longer readily marketed.

A former chief executive officer of a pharmaceutical company that developed and marketed a line of conventional consumer and specialty toothpastes informed this inventor of the difficulties of developing such a product, noting that the limited need for such a product within the general population was another likely reason for it not being developed to date. Thus, there is a long-felt need in the market for oral care products, such as a toothpaste or similar product, that a hunter can use for personal care, but which will not have an odor that prey will perceive as being unusual, and potentially cause it to flee.

A review of prior art references generally disclose dentifrice products containing odor-eliminating agents. For example, Johnson, in U.S. Patent Application Publication No. U.S. Ser. No. 2001/0031220 A1 (“the '220 application”) and U.S. Pat. No. 6,340,447 B2 discloses using a mouthwash containing up to 1% by weight of activated carbon as part of a regimen for a hunter to use to reduce bodily scents prior to hunting. The '220 application discloses using a mouthwash containing up to 1% (by weight) activated carbon, or activated charcoal as a substitute, prior to hunting, to block odors. In published patent application U.S. Ser. No. 2002/0146383, (“the '383 application”) Johnson discloses using a zeolite to reduce a hunter's scent. The use of a zeolite instead of activated carbon “overcomes the smutting problem that exists when activated carbon is sprayed on hunter's apparel” (¶34), and, by implication, when used in a mouthwash. Aside from a mouthwash, the Johnson references make no suggestion about other oral care products.

Donchi (U.S. Pat. No. 1,716,035) discloses using the combination of an activated vegetable carbon material in conjunction with keiselguhr (an absorbent agent) in a toothpaste or toothpowder formulation that contains sodium bicarbonate, saccharin, and flavorings such as oil of wintergreen and oil of peppermint.

Fehr (U.S. Pat. No. 173,607) discloses a deodorizing and disinfecting toothpowder which may be plain or perfumed, in which magnesium silicate and salicylic acid are mixed together in a solvent such as alcohol, and then dried.

In U.S. Pat. No. 3,943,240 (“the '240 patent) Delaney et al. disclose a general dental cream and toothpaste formulations, wherein sodium bicarbonate is the active agent. In a related patent (U.S. Pat. No. 3,935,305, “the '305 patent”) Delaney et al. disclose a similar toothpaste formulation, in which calcium carbonate is the principal abrasive and a lesser amount of sodium bicarbonate is an additional abrasive. In addition, the formulations may include titanium dioxide, sodium lauryl sulphate, sodium saccharin, water, glycerol and flavorings. Among the flavorings that could be added are oil or peppermint, spearmint, orange, lemon, cinnamon, and others, but nothing specific is stated about scents or flavorings that would be attractive to animals.

In U.S. Pat. No. 4,181,712 Rialdi discloses a toothpaste formulation comprising a detergent base, a micronized silica gel and activated charcoal mixture in a formulation containing no abrasive agents. Glycyrrhizin can be added as a flavoring, as well as antimicrobial agents.

White (U.S. Pat. No. 5,117,821) discloses a hunting mask for purifying the breath of a hunter, comprising a face mask having a one-way air intake valve, a tube for conducting the hunter's breath away from the mask to an intake end of an air purification canister, and a one-way discharge valve at the discharge end of the canister for controlled passage of breath to ambient atmosphere.

Thus, the focus of many references has been the elimination of scents from the hunter. None of these references suggest using scents to attract the prey animal towards the hunter's position. However, it would appear that the problems of these references could be resolved by enabling the hunter to maintain their normal oral care regimen, without the introduction of odors or scents, as achieved by using an embodiment of the present invention.

### BRIEF SUMMARY OF THE INVENTION

An object of the present invention is to provide an oral care product for use by hunters whose composition comprises agents containing no added odors or scents.

Another object of the present invention is to provide a method of oral care for a hunter, comprising applying a quantity of a dentifrice, such as a toothpaste, to the teeth, using a composition that has no added scents or odors.

Still another object of the present invention is to provide an oral care product for use by hunters whose composition comprises agents containing added odors or scents which may either be attractive to animals, or which odors or scents would not be recognized by such animals as being offensive to the animal.

Another object of the present invention is to provide a method of oral care for a hunter, comprising applying a quantity of a dentifrice, such as a toothpaste, to the teeth, using a composition that has comprises agents containing added odors or scents which may either be attractive to animals, or which odors or scents would not be recognized by such animals as being offensive to the animal.

The present invention is a method of preparing for hunting which comprises applying a quantity of a toothpaste to the hunter's teeth, the toothpaste formulated to contain no added scents or odors. The formulation provides hunters with the ability to practice their normal tooth brushing routine, but without adding a scent, which if detected by the prey animal, could compromise the hunter's objective of being scent free to avoid detection by the prey while hunting. An embodiment comprises a mixture of calcium carbonate, a polyol; water; sodium bicarbonate; sodium lauryl sulfate; titanium dioxide; guar gum, and sodium saccharin.

### DETAILED DESCRIPTION OF THE INVENTION

Understanding the special needs of the avid deer hunter, there is a long-felt need for a toothpaste or other oral care product made using a scent-free formulation. The majority of conventional toothpaste formulations are soap based, with the added scents and flavorings being necessary to mask the unfavorable soap taste.

The Formulation of the Present Invention Meets the Following Criteria.

It is a baking soda based product (sodium bicarbonate), which effects a natural mouth odor elimination; has no soap taste; a sweetener is added for a tolerable taste; it has no obvious smell; no Food and Drug Administration (“FDA”) approval is needed because the composition has been developed using known and approved materials; it is manufactured in an FDA approved facility; and no dental product claims are being made that would require FDA approval. No fluoride is added in one embodiment, thereby addressing certain regulatory labeling issues. Other embodiments could include fluoride if necessary.

Thus, a hunter can maintain their normal routine of tooth care by brushing their teeth, only using the toothpaste of the present invention prior to going out hunting, or even while in the field. For purposes of the present specification, brushing one's teeth is also intended to the include brushing of dentures, bridges, dental implants, or other dental prostheses, and can also include brushing the gums and/or the tongue, as commonly recommended by dental professionals. Because there is no added scent or flavoring, there is reduced likelihood of a cloud of odor building up around the hunter which would alert a prey animal to a “non-natural” scent in its environment.

With a knowledge of a prey animal's favorite food, or foods that are appropriate to a given season of the year, an embodiment of the present invention can be formulated to attract the animal towards the hunter. For example, among the favorite foods of white tail deer are acorns, apples, cranberries, beechnuts, corn, wheat, rye and a variety of vegetation on which they routinely browse. Extracts pf these could be used either to scent or to flavor an embodiment of the present invention to attract the animal towards the hunter. Similarly, the types of salts that are found in salt licks could also be incorporated into oral care products, in order to attract the prey towards the hunter. As another example, and not intended to be a limitation, brown bears are attracted to salmonberries and such a flavoring could be added for use by bear hunters in certain regions. These flavorings could be used in concentrations ranging from approximately 0.01% to about 5% by weight of the formulation. Other scents or flavorings could also be added to the formulation, which may not be appealing to humans, may be recognized by the particular prey animal as one that is typical of its environment, and thus, would not indicate to the animal the presence of a potential threat, such as a hunter. This could include odors characteristic of the animal's favorite vegetation, a forest odor, or scent of other animals known to be acceptable to the prey.

While much of the present specification has been written based on the behavior of the North American white-tailed deer, it is to be understood that this is merely by way of illustration. Embodiments of the present invention can be used for other members of the deer family, such as, but not intended to be limited to, caribou, elk, moose and other antlered species. Embodiments of the present invention could also be used when hunting other animals, regardless of the environment or continent (i.e.,) whether in North America, central Africa, southern Asia and the like.

### EXAMPLE 1

### Toothpaste Formulation

The toothpaste formulation of the present invention has the following formulation:

| | % by weight |
|---|---|
| Calcium Carbonate (Vicron 15-15) | 20-75 |
| Polyol | 10-30 |
| Water | 25-55 |
| Sodium Bicarbonate #1 | 0.001-40 |
| Sodium Lauryl Sulfate | 0.001-20 |
| Titanium Dioxide | 0.1-10 |
| Guar Gum | 0.02-5 |
| Sodium Saccharin | 0.001-5 |

In addition to its formulation in a toothpaste, the present invention can be formulated as a dental cream, gel, or a toothpowder. Polishing materials are present in solid or pasty oral preparations. While calcium carbonate is the polishing agent in the formulation of the present invention, other materials can be utilized. While not intended to be limited, other examples of suitable polishing materials include sodium metaphosphate, potassium metaphosphate, tricalcium phosphate, dihydrated calcium phosphate, calcium pyrophosphate, magnesium orthophosphate, trimagnesium phosphate, alumina, aluminum silicate, zirconium silicates, silica, bentonite, and mixtures thereof. The polishing materials are generally present in an amount that ranges from about 20% to about 80% by weight of the formulation. In a toothpaste formulation, they are generally present in an amount ranging from about 20% to about 75%.

The polyol can be chosen from the group consisting of glycerol, polyethylene glycol, sorbitol and others known to those skilled in the art.

The water content of toothpaste and dental creams is about 25% to about 55% by weight.

Sweeteners may be selected from among the following compounds:

Water soluble sweeteners such as monosaccharides, disaccharides and polysaccharides, including glucose, ribose mannose, galactose, fructose, sucrose, maltose, partially hydrolyzed starch, corn syrup, sugar alcohols such as mannitol, sorbitol, or xylitol.

Water-soluble artificial sweeteners, such as sodium saccharin, cyclamate salts, aspartyl-phenylalanine methyl ester, acesulfame-K, and the like. The amount of sweetener will vary, and can range from about 0.001% to about 40% by weight of the formulation. Sodium saccharin is used as the sweetener in the formulation of Example 1. Other natural and artificial flavorings can be added, provided that they do not impart any additional scent to the formulation Sodium bicarbonate is present in the range of about 0.001% to about 40% by weight of the formulation.

Sodium bicarbonate is present in the range of about 0.001% to about 40% by weight of the formulation.

Sodium lauryl sulfate is present in the range of 0.001% to about 20% by weight of the formulation. Other surfactants known to those skilled in the art may also be utilized in the formulation of the present invention.

Thickeners or gelling agents can be selected from guar gum, as used in the present invention, or gum tragacanth, xanthan gum, pectin, carrageenans, agarose, alginates or starch. Modified celluloses, such as carboxymethyl cellulose, ethyl cellulose, methylcellulose could also be employed.

Fluoride-containing compounds may be added to the formulation of the present invention. Exemplary fluorine-providing compounds are inorganic fluoride salts such as soluble alkali metals, alkaline earth metals, and heavy metal salts, such as sodium fluoride, potassium fluoride, ammonium fluoride, cuprous fluoride, zinc fluoride, stannic fluoride, stannous fluoride, barium fluoride, sodium fluorosilicate, ammonium fluorosilicate, sodium fluorozirconate, sodium monofluorophosphate, aluminum mono- and difluorophosphates, and fluorinated sodium calcium pyrophosphate.

Colorants are often added to oral care formulations, and when present, include any of the dyes approved for use in food drug and cosmetic applications, as known to those skilled in the art, and which will not be further enumerated herein. As described above, the present invention contains no added colorant.

The present invention is a toothpaste with no added scents, which effects a natural short-term mouth odor control. In addition, other agents can be added to the base formulation to provide antiplaque and calculus control, such as chlorhexidine or pyrophosphates.

Although reference is made herein to deer hunters, no such limitation is intended, because the product can be used by hunters or other individuals who are concerned about emitting odors that prey may consider as being “unnatural” to the environment.

Therefore, although the present invention has been described with a certain degree of particularity, it is to be understood that the present disclosure is made by way of illustration, and that numerous changes in the details of composition and concentration may be resorted to without departing from the spirit and scope of the invention.

I claim:

1. A method to prepare for hunting, the method comprising applying an amount of a dentifrice to the teeth before hunting, the dentifrice comprising agents having no added scent.

2. The method as described in claim 1, wherein the dentifrice is a toothpaste.

3. A method to prepare for hunting, the method comprising brushing one's teeth with an effective amount of a toothpaste to reduce odor of the hunter, the toothpaste formulated to include no added scent that would be considered as “unnatural” by a hunter's prey.

4. The method as described in claim 3, wherein the dentifrice is a toothpaste.

5. The method as described in claim 3, wherein the dentifrice is a toothpowder.

6. A method of reducing odor from a hunter, the method comprising the step of applying an effective amount of a toothpaste to the teeth, to reduce odor of the hunter, the toothpaste formulated to include no added scent.

7. The method as described in claim 4, wherein the toothpaste comprises, as percentage by weight, calcium carbonate, 20-75; a polyol, 10-30; water, 25-55; sodium bicarbonate #1, 0.001-40; sodium lauryl sulfate, 0.001-20; titanium dioxide, 0.02-5; a thickener, 0.02-5; and sodium saccharin, 0.001-5.

8. A method to attract an animal towards a hunter, the method comprising the step of applying a dentifrice to the teeth of the hunter, the dentifrice having a quantity of a scent or flavoring favored by the animal, the quantity being effective to attract the animal.

9 The method as described in claim 8, wherein the flavoring is chosen from the group consisting of apple, cranberry, beechnut, acorn, salmonberry, and salt.

10. A toothpaste for hunters to prevent scent, the toothpaste comprising an admixture of calcium carbonate; a polyol; water; sodium bicarbonate; sodium lauryl sulfate; titanium dioxide; a thickener; and a sweetener.

11. The toothpaste as described in claim 10, wherein the sweetener is selected from the group consisting of glucose, fructose, sucrose, mannitol, xylitol, erythritol, sodium cyclamate. sodium saccharin, sorbitol. Acesulfame-K, and aspartyl-phenylalanine methyl ester.

12. The toothpaste as described in claim 10, wherein the thickener is selected from the group consisting of guar gum, carageenan, gum acacia, gum karaya, and gum tragacanth.

13. A method to prepare for hunting, the method comprising the step of applying a dentifrice to the teeth of the hunter, the dentifrice having a quantity of a scent or flavoring sufficient for the scent or flavoring to be recognized by an animal as not representing a threat to the animal.

14. The method as described in claim 13, wherein the dentifrice is a toothpaste, the toothpaste comprising an admixture of calcium carbonate; a polyol; water; sodium bicarbonate; sodium lauryl sulfate; titanium dioxide; a thickener; and a flavoring.
